SEQUENCE LISTING (14 sequences)

SEQ ID NO: 1 - DNA, 3480 bp, Phaeodactylum tricornutum
accgaaaacatttgggccagcataatctgtgacccatagaaaattgttcgcctagctttttctaatagtgtggttctttccttttcgtgtcggtctcttgccgcatcatcgtctactcactgtcgagcgaaagctgacagtgagactcgttcacattgagtcacggacttgctcggtttacgccgcgtcgatgacaacaaccgttaaaaattactaaaaacgttataatcacggaagaattgtccataaagtactgtgactacgacacattgagagatccacaaagcccatgttttccggcggatggcggtatttgtcgtttgctgcgacaccagctgctgtgtccgaaaaaagacacgtaatgacgatccggactcacagtcaaaaaggccaaccggatttgggagagtgaagcctttttgggggatgtcgtgctttttcgcgtgatgaaacaggtgatgtttcccggaccagaacgtcgtgtcacttttccccttctttgcgttccgtcgtcaaaacttgccttctggcctctcttcatctgtgcgcacccaaacagaagccaagctttttctgcatatctttggctgacgaagctcccgttgttttgttttgtgccggtgtgcggttgtaaaaggatcttgtcgtcagtcaaccaaaccatgagcagtggtgccgacaatagacagtcgacctcttcggagttgcatacgcaccgcctgcctcggaacgcggaactggaagaagtatttgccaaagtttgggaagagtaagtgctatctatcgttgtattttgcagaatacaccaaatgttgattggacgcacactgtggagacagatactccggtcggcttctttcctggtatcttactgacgaatcttgatgctatttgctttcttctttcgccaacaacgaaatataacaggcttgtcaagattgcgtgtgatcacttgcctattcttcaggatgtaaccatgcaaatcgtagacactgtgttcgtcccgaaggtgatcgcgaagcagctcatgctcctcgtcgcgctcgaaacgggtatgatgacggcccggcaaatcacctcgattatacaatctgttttgacgaacttcagtagccgctcccggctcatccgacaactcgagtcggaacagcaacaagctagcgttcaggacgagtggatggatctagcggagcgcattgacagcatccaggggaacgatgtatggcggtccgacccgagctgccccttgtacgaacaggaacgtatttcggcgcggattgacgaattggtgcaccttatgcgacgccgggatatctttgaactcatgtttgtgctgcgtgcttcgattggacgcaacaagtttgggctgttgcacgaagggctgttcagcaaggcgttagcgggtaccaaagtactcgtcgagacgtatcacaacgtggtctgtgctgccttggacttttgttgcgacgctcccgtgtctcctgacgaggatcctatccctacggatgcccgtctcgcatttttcaacgaaactcgacacgcttacggacgtacagccttgctcctatccggtggtgcggccttgggcttttaccatactggtgtcgtcaagacgcttatggaaaatcgcctcatgccgcgtgtaattggtggcagctcggctggttcgctcgtgtgtgccatgatagctacacggacagacgaagaatgcgtgcacgacatgttcaacgcccaaggtaccgacgctccgggacattccggccaactcccactcaactttttccgccccctccaaacggggaatatcaacgcagccaaggtagacgcaacgccgaacaaacaactcgggggaattcgtgaagtgtactacaatactgctggattctttcacgatgccaagcggaccttgcaagggttggttccaattcctctccgacacttttccgccgtgttgtacgatatcgtcaccggcaaccgtcggcctcaagacatgctcatgaatgatacagaacacttccgagcttgcgtacgggcctgtgtcggtaacttcacatttcaagaagctttcgaccggactgggcgtattctgaacatcgtggtgacgcccaagaacaattccgatccgccccgcttactcaactacttgacggcaccgcacgttatggtatggtcagctgccgtcgcgagctcctccctacccggagtttttgaagctaatcgactagttgtcaaggaagcagacggttgggaacggtacgagtcgggcggcgcgccacagcacttttcggatggatcgatggaacaggatttgcccatgcagcagctatcggagatgttcaacgtcaaccactttttgatctcgcaggccaacccacacgccgtcatgtttgccaattatcaacaaaagaattcggtgtggagtaatcctgttacgggctttgtggattctattctgacctttttacgcgatcaagtgcgcacttggttgttgcatctcgtggcgtgcgttggcgctcgtagtattacacctatgttccagactcaacgtggaattggtacaactttcctgacgcaagaatacgaagggcggtcttgcgacatttcactcatcccatggttgggtcatcggggactcttcagtgccttattgcacattatctacaacccaagggaagccgagtttcgcgaatggatccaagcagctgaacgagaaacctggcgacacattccggccatcaaatcgcacatcgccgaagaagttactctggatcgttgtgtacaaaggctacgaaaaagactagtgcaggaatcttgggaaaagcacaccagtggggtcgaacaggagacaatgggagggcgagttcccagtttctttacaagcactagtctcgtgaatttaagcggtcttaacgtatcggaccaaccagcagtggaaggattgcacggatacaatcgaaattccggctctgatgttcggaaagtagtcgcaagcccgatgaatattgtacccgatataaatattcattcgggctggggcggacaaggtctgagaggtaatcggtctagcggaaatttgcagcggtcgatctccgacggatcgggtctctttattgacgatgaagagcctacttctcagcaacaggcgggtgaagagacagaaatgccgctcagaggccaccttgcgcgaaacagtcaccgcaccaaaagtgaaaacagtagtctacaaactcccggctatgtcaagtcgacgagcatggcgaagttctactatcgcaagactggtgattacaatgttcctgaacttcgcaagacaacaagcgatcccctgattgaaagagagggtgaaagcgattcatcgagtaccgatggtacagcaccgcgcttgtcacatcagcggcggaagtctaaaagtcataccgaactaaactggacttcgtcgcctctcaactgagattggcgaccaatttaaaatagctatat

SEQ ID NO: 2 - DNA, 2789 bp, Phaeodactylum tricornutum
atgagcagtggtgccgacaatagacagtcgacctcttcggagttgcatacgcaccgcctgcctcggaacgcggaactggaagaagtatttgccaaagtttgggaagagtaagtgctatctatcgttgtattttgcagaatacaccaaatgttgattggacgcacactgtggagacagatactccggtcggcttctttcctggtatcttactgacgaatcttgatgctatttgctttcttctttcgccaacaacgaaatataacaggcttgtcaagattgcgtgtgatcacttgcctattcttcaggatgtaaccatgcaaatcgtagacactgtgttcgtcccgaaggtgatcgcgaagcagctcatgctcctcgtcgcgctcgaaacgggtatgatgacggcccggcaaatcacctcgattatacaatctgttttgacgaacttcagtagccgctcccggctcatccgacaactcgagtcggaacagcaacaagctagcgttcaggacgagtggatggatctagcggagcgcattgacagcatccaggggaacgatgtatggcggtccgacccgagctgccccttgtacgaacaggaacgtatttcggcgcggattgacgaattggtgcaccttatgcgacgccgggatatctttgaactcatgtttgtgctgcgtgcttcgattggacgcaacaagtttgggctgttgcacgaagggctgttcagcaaggcgttagcgggtaccaaagtactcgtcgagacgtatcacaacgtggtctgtgctgccttggacttttgttgcgacgctcccgtgtctcctgacgaggatcctatccctacggatgcccgtctcgcatttttcaacgaaactcgacacgcttacggacgtacagccttgctcctatccggtggtgcggccttgggcttttaccatactggtgtcgtcaagacgcttatggaaaatcgcctcatgccgcgtgtaattggtggcagctcggctggttcgctcgtgtgtgccatgatagctacacggacagacgaagaatgcgtgcacgacatgttcaacgcccaaggtaccgacgctccgggacattccggccaactcccactcaactttttccgccccctccaaacggggaatatcaacgcagccaaggtagacgcaacgccgaacaaacaactcgggggaattcgtgaagtgtactacaatactgctggattctttcacgatgccaagcggaccttgcaagggttggttccaattcctctccgacacttttccgccgtgttgtacgatatcgtcaccggcaaccgtcggcctcaagacatgctcatgaatgatacagaacacttccgagcttgcgtacgggcctgtgtcggtaacttcacatttcaagaagctttcgaccggactgggcgtattctgaacatcgtggtgacgcccaagaacaattccgatccgccccgcttactcaactacttgacggcaccgcacgttatggtatggtcagctgccgtcgcgagctcctccctacccggagtttttgaagctaatcgactagttgtcaaggaagcagacggttgggaacggtacgagtcgggcggcgcgccacagcacttttcggatggatcgatggaacaggatttgcccatgcagcagctatcggagatgttcaacgtcaaccactttttgatctcgcaggccaacccacacgccgtcatgtttgccaattatcaacaaaagaattcggtgtggagtaatcctgttacgggctttgtggattctattctgacctttttacgcgatcaagtgcgcacttggttgttgcatctcgtggcgtgcgttggcgctcgtagtattacacctatgttccagactcaacgtggaattggtacaactttcctgacgcaagaatacgaagggcggtcttgcgacatttcactcatcccatggttgggtcatcggggactcttcagtgccttattgcacattatctacaacccaagggaagccgagtttcgcgaatggatccaagcagctgaacgagaaacctggcgacacattccggccatcaaatcgcacatcgccgaagaagttactctggatcgttgtgtacaaaggctacgaaaaagactagtgcaggaatcttgggaaaagcacaccagtggggtcgaacaggagacaatgggagggcgagttcccagtttctttacaagcactagtctcgtgaatttaagcggtcttaacgtatcggaccaaccagcagtggaaggattgcacggatacaatcgaaattccggctctgatgttcggaaagtagtcgcaagcccgatgaatattgtacccgatataaatattcattcgggctggggcggacaaggtctgagaggtaatcggtctagcggaaatttgcagcggtcgatctccgacggatcgggtctctttattgacgatgaagagcctacttctcagcaacaggcgggtgaagagacagaaatgccgctcagaggccaccttgcgcgaaacagtcaccgcaccaaaagtgaaaacagtagtctacaaactcccggctatgtcaagtcgacgagcatggcgaagttctactatcgcaagactggtgattacaatgttcctgaacttcgcaagacaacaagcgatcccctgattgaaagagagggtgaaagcgattcatcgagtaccgatggtacagcaccgcgcttgtcacatcagcggcggaagtctaaaagtcataccgaactaaactggacttcgtcgcctctcaactga

SEQ ID NO: 3 - DNA, 2483 bp, Phaeodactylum tricornutum
atgtaaccatgcaaatcgtagacactgtgttcgtcccgaaggtgatcgcgaagcagctcatgctcctcgtcgcgctcgaaacgggtatgatgacggcccggcaaatcacctcgattatacaatctgttttgacgaacttcagtagccgctcccggctcatccgacaactcgagtcggaacagcaacaagctagcgttcaggacgagtggatggatctagcggagcgcattgacagcatccaggggaacgatgtatggcggtccgacccgagctgccccttgtacgaacaggaacgtatttcggcgcggattgacgaattggtgcaccttatgcgacgccgggatatctttgaactcatgtttgtgctgcgtgcttcgattggacgcaacaagtttgggctgttgcacgaagggctgttcagcaaggcgttagcgggtaccaaagtactcgtcgagacgtatcacaacgtggtctgtgctgccttggacttttgttgcgacgctcccgtgtctcctgacgaggatcctatccctacggatgcccgtctcgcatttttcaacgaaactcgacacgcttacggacgtacagccttgctcctatccggtggtgcggccttgggcttttaccatactggtgtcgtcaagacgcttatggaaaatcgcctcatgccgcgtgtaattggtggcagctcggctggttcgctcgtgtgtgccatgatagctacacggacagacgaagaatgcgtgcacgacatgttcaacgcccaaggtaccgacgctccgggacattccggccaactcccactcaactttttccgccccctccaaacggggaatatcaacgcagccaaggtagacgcaacgccgaacaaacaactcgggggaattcgtgaagtgtactacaatactgctggattctttcacgatgccaagcggaccttgcaagggttggttccaattcctctccgacacttttccgccgtgttgtacgatatcgtcaccggcaaccgtcggcctcaagacatgctcatgaatgatacagaacacttccgagcttgcgtacgggcctgtgtcggtaacttcacatttcaagaagctttcgaccggactgggcgtattctgaacatcgtggtgacgcccaagaacaattccgatccgccccgcttactcaactacttgacggcaccgcacgttatggtatggtcagctgccgtcgcgagctcctccctacccggagtttttgaagctaatcgactagttgtcaaggaagcagacggttgggaacggtacgagtcgggcggcgcgccacagcacttttcggatggatcgatggaacaggatttgcccatgcagcagctatcggagatgttcaacgtcaaccactttttgatctcgcaggccaacccacacgccgtcatgtttgccaattatcaacaaaagaattcggtgtggagtaatcctgttacgggctttgtggattctattctgacctttttacgcgatcaagtgcgcacttggttgttgcatctcgtggcgtgcgttggcgctcgtagtattacacctatgttccagactcaacgtggaattggtacaactttcctgacgcaagaatacgaagggcggtcttgcgacatttcactcatcccatggttgggtcatcggggactcttcagtgccttattgcacattatctacaacccaagggaagccgagtttcgcgaatggatccaagcagctgaacgagaaacctggcgacacattccggccatcaaatcgcacatcgccgaagaagttactctggatcgttgtgtacaaaggctacgaaaaagactagtgcaggaatcttgggaaaagcacaccagtggggtcgaacaggagacaatgggagggcgagttcccagtttctttacaagcactagtctcgtgaatttaagcggtcttaacgtatcggaccaaccagcagtggaaggattgcacggatacaatcgaaattccggctctgatgttcggaaagtagtcgcaagcccgatgaatattgtacccgatataaatattcattcgggctggggcggacaaggtctgagaggtaatcggtctagcggaaatttgcagcggtcgatctccgacggatcgggtctctttattgacgatgaagagcctacttctcagcaacaggcgggtgaagagacagaaatgccgctcagaggccaccttgcgcgaaacagtcaccgcaccaaaagtgaaaacagtagtctacaaactcccggctatgtcaagtcgacgagcatggcgaagttctactatcgcaagactggtgattacaatgttcctgaacttcgcaagacaacaagcgatcccctgattgaaagagagggtgaaagcgattcatcgagtaccgatggtacagcaccgcgcttgtcacatcagcggcggaagtctaaaagtcataccgaactaaactggacttcgtcgcctctcaactga

SEQ ID NO: 4 - DNA, 2475 bp, Phaeodactylum tricornutum
atgcaaatcgtagacactgtgttcgtcccgaaggtgatcgcgaagcagctcatgctcctcgtcgcgctcgaaacgggtatgatgacggcccggcaaatcacctcgattatacaatctgttttgacgaacttcagtagccgctcccggctcatccgacaactcgagtcggaacagcaacaagctagcgttcaggacgagtggatggatctagcggagcgcattgacagcatccaggggaacgatgtatggcggtccgacccgagctgccccttgtacgaacaggaacgtatttcggcgcggattgacgaattggtgcaccttatgcgacgccgggatatctttgaactcatgtttgtgctgcgtgcttcgattggacgcaacaagtttgggctgttgcacgaagggctgttcagcaaggcgttagcgggtaccaaagtactcgtcgagacgtatcacaacgtggtctgtgctgccttggacttttgttgcgacgctcccgtgtctcctgacgaggatcctatccctacggatgcccgtctcgcatttttcaacgaaactcgacacgcttacggacgtacagccttgctcctatccggtggtgcggccttgggcttttaccatactggtgtcgtcaagacgcttatggaaaatcgcctcatgccgcgtgtaattggtggcagctcggctggttcgctcgtgtgtgccatgatagctacacggacagacgaagaatgcgtgcacgacatgttcaacgcccaaggtaccgacgctccgggacattccggccaactcccactcaactttttccgccccctccaaacggggaatatcaacgcagccaaggtagacgcaacgccgaacaaacaactcgggggaattcgtgaagtgtactacaatactgctggattctttcacgatgccaagcggaccttgcaagggttggttccaattcctctccgacacttttccgccgtgttgtacgatatcgtcaccggcaaccgtcggcctcaagacatgctcatgaatgatacagaacacttccgagcttgcgtacgggcctgtgtcggtaacttcacatttcaagaagctttcgaccggactgggcgtattctgaacatcgtggtgacgcccaagaacaattccgatccgccccgcttactcaactacttgacggcaccgcacgttatggtatggtcagctgccgtcgcgagctcctccctacccggagtttttgaagctaatcgactagttgtcaaggaagcagacggttgggaacggtacgagtcgggcggcgcgccacagcacttttcggatggatcgatggaacaggatttgcccatgcagcagctatcggagatgttcaacgtcaaccactttttgatctcgcaggccaacccacacgccgtcatgtttgccaattatcaacaaaagaattcggtgtggagtaatcctgttacgggctttgtggattctattctgacctttttacgcgatcaagtgcgcacttggttgttgcatctcgtggcgtgcgttggcgctcgtagtattacacctatgttccagactcaacgtggaattggtacaactttcctgacgcaagaatacgaagggcggtcttgcgacatttcactcatcccatggttgggtcatcggggactcttcagtgccttattgcacattatctacaacccaagggaagccgagtttcgcgaatggatccaagcagctgaacgagaaacctggcgacacattccggccatcaaatcgcacatcgccgaagaagttactctggatcgttgtgtacaaaggctacgaaaaagactagtgcaggaatcttgggaaaagcacaccagtggggtcgaacaggagacaatgggagggcgagttcccagtttctttacaagcactagtctcgtgaatttaagcggtcttaacgtatcggaccaaccagcagtggaaggattgcacggatacaatcgaaattccggctctgatgttcggaaagtagtcgcaagcccgatgaatattgtacccgatataaatattcattcgggctggggcggacaaggtctgagaggtaatcggtctagcggaaatttgcagcggtcgatctccgacggatcgggtctctttattgacgatgaagagcctacttctcagcaacaggcgggtgaagagacagaaatgccgctcagaggccaccttgcgcgaaacagtcaccgcaccaaaagtgaaaacagtagtctacaaactcccggctatgtcaagtcgacgagcatggcgaagttctactatcgcaagactggtgattacaatgttcctgaacttcgcaagacaacaagcgatcccctgattgaaagagagggtgaaagcgattcatcgagtaccgatggtacagcaccgcgcttgtcacatcagcggcggaagtctaaaagtcataccgaactaaactggacttcgtcgcctctcaactga

SEQ ID NO: 5 - DNA, 2424 bp, Phaeodactylum tricornutum
atgctcctcgtcgcgctcgaaacgggtatgatgacggcccggcaaatcacctcgattatacaatctgttttgacgaacttcagtagccgctcccggctcatccgacaactcgagtcggaacagcaacaagctagcgttcaggacgagtggatggatctagcggagcgcattgacagcatccaggggaacgatgtatggcggtccgacccgagctgccccttgtacgaacaggaacgtatttcggcgcggattgacgaattggtgcaccttatgcgacgccgggatatctttgaactcatgtttgtgctgcgtgcttcgattggacgcaacaagtttgggctgttgcacgaagggctgttcagcaaggcgttagcgggtaccaaagtactcgtcgagacgtatcacaacgtggtctgtgctgccttggacttttgttgcgacgctcccgtgtctcctgacgaggatcctatccctacggatgcccgtctcgcatttttcaacgaaactcgacacgcttacggacgtacagccttgctcctatccggtggtgcggccttgggcttttaccatactggtgtcgtcaagacgcttatggaaaatcgcctcatgccgcgtgtaattggtggcagctcggctggttcgctcgtgtgtgccatgatagctacacggacagacgaagaatgcgtgcacgacatgttcaacgcccaaggtaccgacgctccgggacattccggccaactcccactcaactttttccgccccctccaaacggggaatatcaacgcagccaaggtagacgcaacgccgaacaaacaactcgggggaattcgtgaagtgtactacaatactgctggattctttcacgatgccaagcggaccttgcaagggttggttccaattcctctccgacacttttccgccgtgttgtacgatatcgtcaccggcaaccgtcggcctcaagacatgctcatgaatgatacagaacacttccgagcttgcgtacgggcctgtgtcggtaacttcacatttcaagaagctttcgaccggactgggcgtattctgaacatcgtggtgacgcccaagaacaattccgatccgccccgcttactcaactacttgacggcaccgcacgttatggtatggtcagctgccgtcgcgagctcctccctacccggagtttttgaagctaatcgactagttgtcaaggaagcagacggttgggaacggtacgagtcgggcggcgcgccacagcacttttcggatggatcgatggaacaggatttgcccatgcagcagctatcggagatgttcaacgtcaaccactttttgatctcgcaggccaacccacacgccgtcatgtttgccaattatcaacaaaagaattcggtgtggagtaatcctgttacgggctttgtggattctattctgacctttttacgcgatcaagtgcgcacttggttgttgcatctcgtggcgtgcgttggcgctcgtagtattacacctatgttccagactcaacgtggaattggtacaactttcctgacgcaagaatacgaagggcggtcttgcgacatttcactcatcccatggttgggtcatcggggactcttcagtgccttattgcacattatctacaacccaagggaagccgagtttcgcgaatggatccaagcagctgaacgagaaacctggcgacacattccggccatcaaatcgcacatcgccgaagaagttactctggatcgttgtgtacaaaggctacgaaaaagactagtgcaggaatcttgggaaaagcacaccagtggggtcgaacaggagacaatgggagggcgagttcccagtttctttacaagcactagtctcgtgaatttaagcggtcttaacgtatcggaccaaccagcagtggaaggattgcacggatacaatcgaaattccggctctgatgttcggaaagtagtcgcaagcccgatgaatattgtacccgatataaatattcattcgggctggggcggacaaggtctgagaggtaatcggtctagcggaaatttgcagcggtcgatctccgacggatcgggtctctttattgacgatgaagagcctacttctcagcaacaggcgggtgaagagacagaaatgccgctcagaggccaccttgcgcgaaacagtcaccgcaccaaaagtgaaaacagtagtctacaaactcccggctatgtcaagtcgacgagcatggcgaagttctactatcgcaagactggtgattacaatgttcctgaacttcgcaagacaacaagcgatcccctgattgaaagagagggtgaaagcgattcatcgagtaccgatggtacagcaccgcgcttgtcacatcagcggcggaagtctaaaagtcataccgaactaaactggacttcgtcgcctctcaactga

SEQ ID NO: 6 - DNA, 2397 bp, Phaeodactylum tricornutum
atgatgacggcccggcaaatcacctcgattatacaatctgttttgacgaacttcagtagccgctcccggctcatccgacaactcgagtcggaacagcaacaagctagcgttcaggacgagtggatggatctagcggagcgcattgacagcatccaggggaacgatgtatggcggtccgacccgagctgccccttgtacgaacaggaacgtatttcggcgcggattgacgaattggtgcaccttatgcgacgccgggatatctttgaactcatgtttgtgctgcgtgcttcgattggacgcaacaagtttgggctgttgcacgaagggctgttcagcaaggcgttagcgggtaccaaagtactcgtcgagacgtatcacaacgtggtctgtgctgccttggacttttgttgcgacgctcccgtgtctcctgacgaggatcctatccctacggatgcccgtctcgcatttttcaacgaaactcgacacgcttacggacgtacagccttgctcctatccggtggtgcggccttgggcttttaccatactggtgtcgtcaagacgcttatggaaaatcgcctcatgccgcgtgtaattggtggcagctcggctggttcgctcgtgtgtgccatgatagctacacggacagacgaagaatgcgtgcacgacatgttcaacgcccaaggtaccgacgctccgggacattccggccaactcccactcaactttttccgccccctccaaacggggaatatcaacgcagccaaggtagacgcaacgccgaacaaacaactcgggggaattcgtgaagtgtactacaatactgctggattctttcacgatgccaagcggaccttgcaagggttggttccaattcctctccgacacttttccgccgtgttgtacgatatcgtcaccggcaaccgtcggcctcaagacatgctcatgaatgatacagaacacttccgagcttgcgtacgggcctgtgtcggtaacttcacatttcaagaagctttcgaccggactgggcgtattctgaacatcgtggtgacgcccaagaacaattccgatccgccccgcttactcaactacttgacggcaccgcacgttatggtatggtcagctgccgtcgcgagctcctccctacccggagtttttgaagctaatcgactagttgtcaaggaagcagacggttgggaacggtacgagtcgggcggcgcgccacagcacttttcggatggatcgatggaacaggatttgcccatgcagcagctatcggagatgttcaacgtcaaccactttttgatctcgcaggccaacccacacgccgtcatgtttgccaattatcaacaaaagaattcggtgtggagtaatcctgttacgggctttgtggattctattctgacctttttacgcgatcaagtgcgcacttggttgttgcatctcgtggcgtgcgttggcgctcgtagtattacacctatgttccagactcaacgtggaattggtacaactttcctgacgcaagaatacgaagggcggtcttgcgacatttcactcatcccatggttgggtcatcggggactcttcagtgccttattgcacattatctacaacccaagggaagccgagtttcgcgaatggatccaagcagctgaacgagaaacctggcgacacattccggccatcaaatcgcacatcgccgaagaagttactctggatcgttgtgtacaaaggctacgaaaaagactagtgcaggaatcttgggaaaagcacaccagtggggtcgaacaggagacaatgggagggcgagttcccagtttctttacaagcactagtctcgtgaatttaagcggtcttaacgtatcggaccaaccagcagtggaaggattgcacggatacaatcgaaattccggctctgatgttcggaaagtagtcgcaagcccgatgaatattgtacccgatataaatattcattcgggctggggcggacaaggtctgagaggtaatcggtctagcggaaatttgcagcggtcgatctccgacggatcgggtctctttattgacgatgaagagcctacttctcagcaacaggcgggtgaagagacagaaatgccgctcagaggccaccttgcgcgaaacagtcaccgcaccaaaagtgaaaacagtagtctacaaactcccggctatgtcaagtcgacgagcatggcgaagttctactatcgcaagactggtgattacaatgttcctgaacttcgcaagacaacaagcgatcccctgattgaaagagagggtgaaagcgattcatcgagtaccgatggtacagcaccgcgcttgtcacatcagcggcggaagtctaaaagtcataccgaactaaactggacttcgtcgcctctcaactga

SEQ ID NO: 7 - DNA, 2394 bp, Phaeodactylum tricornutum
atgacggcccggcaaatcacctcgattatacaatctgttttgacgaacttcagtagccgctcccggctcatccgacaactcgagtcggaacagcaacaagctagcgttcaggacgagtggatggatctagcggagcgcattgacagcatccaggggaacgatgtatggcggtccgacccgagctgccccttgtacgaacaggaacgtatttcggcgcggattgacgaattggtgcaccttatgcgacgccgggatatctttgaactcatgtttgtgctgcgtgcttcgattggacgcaacaagtttgggctgttgcacgaagggctgttcagcaaggcgttagcgggtaccaaagtactcgtcgagacgtatcacaacgtggtctgtgctgccttggacttttgttgcgacgctcccgtgtctcctgacgaggatcctatccctacggatgcccgtctcgcatttttcaacgaaactcgacacgcttacggacgtacagccttgctcctatccggtggtgcggccttgggcttttaccatactggtgtcgtcaagacgcttatggaaaatcgcctcatgccgcgtgtaattggtggcagctcggctggttcgctcgtgtgtgccatgatagctacacggacagacgaagaatgcgtgcacgacatgttcaacgcccaaggtaccgacgctccgggacattccggccaactcccactcaactttttccgccccctccaaacggggaatatcaacgcagccaaggtagacgcaacgccgaacaaacaactcgggggaattcgtgaagtgtactacaatactgctggattctttcacgatgccaagcggaccttgcaagggttggttccaattcctctccgacacttttccgccgtgttgtacgatatcgtcaccggcaaccgtcggcctcaagacatgctcatgaatgatacagaacacttccgagcttgcgtacgggcctgtgtcggtaacttcacatttcaagaagctttcgaccggactgggcgtattctgaacatcgtggtgacgcccaagaacaattccgatccgccccgcttactcaactacttgacggcaccgcacgttatggtatggtcagctgccgtcgcgagctcctccctacccggagtttttgaagctaatcgactagttgtcaaggaagcagacggttgggaacggtacgagtcgggcggcgcgccacagcacttttcggatggatcgatggaacaggatttgcccatgcagcagctatcggagatgttcaacgtcaaccactttttgatctcgcaggccaacccacacgccgtcatgtttgccaattatcaacaaaagaattcggtgtggagtaatcctgttacgggctttgtggattctattctgacctttttacgcgatcaagtgcgcacttggttgttgcatctcgtggcgtgcgttggcgctcgtagtattacacctatgttccagactcaacgtggaattggtacaactttcctgacgcaagaatacgaagggcggtcttgcgacatttcactcatcccatggttgggtcatcggggactcttcagtgccttattgcacattatctacaacccaagggaagccgagtttcgcgaatggatccaagcagctgaacgagaaacctggcgacacattccggccatcaaatcgcacatcgccgaagaagttactctggatcgttgtgtacaaaggctacgaaaaagactagtgcaggaatcttgggaaaagcacaccagtggggtcgaacaggagacaatgggagggcgagttcccagtttctttacaagcactagtctcgtgaatttaagcggtcttaacgtatcggaccaaccagcagtggaaggattgcacggatacaatcgaaattccggctctgatgttcggaaagtagtcgcaagcccgatgaatattgtacccgatataaatattcattcgggctggggcggacaaggtctgagaggtaatcggtctagcggaaatttgcagcggtcgatctccgacggatcgggtctctttattgacgatgaagagcctacttctcagcaacaggcgggtgaagagacagaaatgccgctcagaggccaccttgcgcgaaacagtcaccgcaccaaaagtgaaaacagtagtctacaaactcccggctatgtcaagtcgacgagcatggcgaagttctactatcgcaagactggtgattacaatgttcctgaacttcgcaagacaacaagcgatcccctgattgaaagagagggtgaaagcgattcatcgagtaccgatggtacagcaccgcgcttgtcacatcagcggcggaagtctaaaagtcataccgaactaaactggacttcgtcgcctctcaactga

SEQ ID NO: 8 - DNA, 2274 bp, Phaeodactylum tricornutum
atggatctagcggagcgcattgacagcatccaggggaacgatgtatggcggtccgacccgagctgccccttgtacgaacaggaacgtatttcggcgcggattgacgaattggtgcaccttatgcgacgccgggatatctttgaactcatgtttgtgctgcgtgcttcgattggacgcaacaagtttgggctgttgcacgaagggctgttcagcaaggcgttagcgggtaccaaagtactcgtcgagacgtatcacaacgtggtctgtgctgccttggacttttgttgcgacgctcccgtgtctcctgacgaggatcctatccctacggatgcccgtctcgcatttttcaacgaaactcgacacgcttacggacgtacagccttgctcctatccggtggtgcggccttgggcttttaccatactggtgtcgtcaagacgcttatggaaaatcgcctcatgccgcgtgtaattggtggcagctcggctggttcgctcgtgtgtgccatgatagctacacggacagacgaagaatgcgtgcacgacatgttcaacgcccaaggtaccgacgctccgggacattccggccaactcccactcaactttttccgccccctccaaacggggaatatcaacgcagccaaggtagacgcaacgccgaacaaacaactcgggggaattcgtgaagtgtactacaatactgctggattctttcacgatgccaagcggaccttgcaagggttggttccaattcctctccgacacttttccgccgtgttgtacgatatcgtcaccggcaaccgtcggcctcaagacatgctcatgaatgatacagaacacttccgagcttgcgtacgggcctgtgtcggtaacttcacatttcaagaagctttcgaccggactgggcgtattctgaacatcgtggtgacgcccaagaacaattccgatccgccccgcttactcaactacttgacggcaccgcacgttatggtatggtcagctgccgtcgcgagctcctccctacccggagtttttgaagctaatcgactagttgtcaaggaagcagacggttgggaacggtacgagtcgggcggcgcgccacagcacttttcggatggatcgatggaacaggatttgcccatgcagcagctatcggagatgttcaacgtcaaccactttttgatctcgcaggccaacccacacgccgtcatgtttgccaattatcaacaaaagaattcggtgtggagtaatcctgttacgggctttgtggattctattctgacctttttacgcgatcaagtgcgcacttggttgttgcatctcgtggcgtgcgttggcgctcgtagtattacacctatgttccagactcaacgtggaattggtacaactttcctgacgcaagaatacgaagggcggtcttgcgacatttcactcatcccatggttgggtcatcggggactcttcagtgccttattgcacattatctacaacccaagggaagccgagtttcgcgaatggatccaagcagctgaacgagaaacctggcgacacattccggccatcaaatcgcacatcgccgaagaagttactctggatcgttgtgtacaaaggctacgaaaaagactagtgcaggaatcttgggaaaagcacaccagtggggtcgaacaggagacaatgggagggcgagttcccagtttctttacaagcactagtctcgtgaatttaagcggtcttaacgtatcggaccaaccagcagtggaaggattgcacggatacaatcgaaattccggctctgatgttcggaaagtagtcgcaagcccgatgaatattgtacccgatataaatattcattcgggctggggcggacaaggtctgagaggtaatcggtctagcggaaatttgcagcggtcgatctccgacggatcgggtctctttattgacgatgaagagcctacttctcagcaacaggcgggtgaagagacagaaatgccgctcagaggccaccttgcgcgaaacagtcaccgcaccaaaagtgaaaacagtagtctacaaactcccggctatgtcaagtcgacgagcatggcgaagttctactatcgcaagactggtgattacaatgttcctgaacttcgcaagacaacaagcgatcccctgattgaaagagagggtgaaagcgattcatcgagtaccgatggtacagcaccgcgcttgtcacatcagcggcggaagtctaaaagtcataccgaactaaactggacttcgtcgcctctcaactga

SEQ ID NO: 9 - DNA, 2234 bp, Phaeodactylum tricornutum
atgtatggcggtccgacccgagctgccccttgtacgaacaggaacgtatttcggcgcggattgacgaattggtgcaccttatgcgacgccgggatatctttgaactcatgtttgtgctgcgtgcttcgattggacgcaacaagtttgggctgttgcacgaagggctgttcagcaaggcgttagcgggtaccaaagtactcgtcgagacgtatcacaacgtggtctgtgctgccttggacttttgttgcgacgctcccgtgtctcctgacgaggatcctatccctacggatgcccgtctcgcatttttcaacgaaactcgacacgcttacggacgtacagccttgctcctatccggtggtgcggccttgggcttttaccatactggtgtcgtcaagacgcttatggaaaatcgcctcatgccgcgtgtaattggtggcagctcggctggttcgctcgtgtgtgccatgatagctacacggacagacgaagaatgcgtgcacgacatgttcaacgcccaaggtaccgacgctccgggacattccggccaactcccactcaactttttccgccccctccaaacggggaatatcaacgcagccaaggtagacgcaacgccgaacaaacaactcgggggaattcgtgaagtgtactacaatactgctggattctttcacgatgccaagcggaccttgcaagggttggttccaattcctctccgacacttttccgccgtgttgtacgatatcgtcaccggcaaccgtcggcctcaagacatgctcatgaatgatacagaacacttccgagcttgcgtacgggcctgtgtcggtaacttcacatttcaagaagctttcgaccggactgggcgtattctgaacatcgtggtgacgcccaagaacaattccgatccgccccgcttactcaactacttgacggcaccgcacgttatggtatggtcagctgccgtcgcgagctcctccctacccggagtttttgaagctaatcgactagttgtcaaggaagcagacggttgggaacggtacgagtcgggcggcgcgccacagcacttttcggatggatcgatggaacaggatttgcccatgcagcagctatcggagatgttcaacgtcaaccactttttgatctcgcaggccaacccacacgccgtcatgtttgccaattatcaacaaaagaattcggtgtggagtaatcctgttacgggctttgtggattctattctgacctttttacgcgatcaagtgcgcacttggttgttgcatctcgtggcgtgcgttggcgctcgtagtattacacctatgttccagactcaacgtggaattggtacaactttcctgacgcaagaatacgaagggcggtcttgcgacatttcactcatcccatggttgggtcatcggggactcttcagtgccttattgcacattatctacaacccaagggaagccgagtttcgcgaatggatccaagcagctgaacgagaaacctggcgacacattccggccatcaaatcgcacatcgccgaagaagttactctggatcgttgtgtacaaaggctacgaaaaagactagtgcaggaatcttgggaaaagcacaccagtggggtcgaacaggagacaatgggagggcgagttcccagtttctttacaagcactagtctcgtgaatttaagcggtcttaacgtatcggaccaaccagcagtggaaggattgcacggatacaatcgaaattccggctctgatgttcggaaagtagtcgcaagcccgatgaatattgtacccgatataaatattcattcgggctggggcggacaaggtctgagaggtaatcggtctagcggaaatttgcagcggtcgatctccgacggatcgggtctctttattgacgatgaagagcctacttctcagcaacaggcgggtgaagagacagaaatgccgctcagaggccaccttgcgcgaaacagtcaccgcaccaaaagtgaaaacagtagtctacaaactcccggctatgtcaagtcgacgagcatggcgaagttctactatcgcaagactggtgattacaatgttcctgaacttcgcaagacaacaagcgatcccctgattgaaagagagggtgaaagcgattcatcgagtaccgatggtacagcaccgcgcttgtcacatcagcggcggaagtctaaaagtcataccgaactaaactggacttcgtcgcctctcaactga

SEQ ID NO: 10 - DNA, 290 bp, Phaeodactylum tricornutum
ggcgttagcgggtaccaaagtactcgtcgagacgtatcacaacgtggtctgtgctgccttggacttttgttgcgacgctcccgtgtctcctgacgaggatcctatccctacggatgcccgtctcgcatttttcaacgaaactcgacacgcttacggacgtacagccttgctcctatccggtggtgcggccttgggcttttaccatactggtgtcgtcaagacgcttatggaaaatcgcctcatgccgcgtgtaattggtggcagctcggctggttcgctcgtgtgtgcca

SEQ ID NO: 11 - DNA, 319 bp, Phaeodactylum tricornutum
ggcgttagcgggtaccaaagtactcgtcgagacgtatcacaacgtggtctgtgctgccttggacttttgttgcgacgctcccgtgtctcctgacgaggatcctatccctacggatgcccgtctcgcatttttcaacgaaactcgacacgcttacggacgtacagccttgctcctatccggtggtgcggccttgggcttttaccatactggtgtcgtcaagacgcttatggaaaatcgcctcatgccgcgtgtaattggtggcagctcggctggttcgctcgtgtgtgccatgatagctacacggacagacgaagaatgc

SEQ ID NO: 12 - DNA, 30 bp, Artificial
  Primer
cttcgtcgacggcgttagcgggtaccaaag

SEQ ID NO: 13 - DNA, 27 bp, Artificial
  Primer
ctttctagatggcacacacgagcgaac

SEQ ID NO: 14 - DNA, 29 bp, Artificial
  Primer
ctttctagagcattcttcgtctgtccgtg

(19) United States
(12) Patent Application Publication
(10) Pub. No.: US 2014/0256927 A1
(43) Pub. Date: Sep. 11, 2014

(54) INCREASING THE LIPID CONTENT IN MICROALGAE BY GENETICALLY MANIPULATING A TRIACYLGLYCEROL (TAG) LIPASE

(71) Applicants: Helge Bode, Oberursel (DE); Claudia Buechel, Mainz (DE); Frederik Barka, Frankfurt (DE); Wolfram Lorenzen, Frankfurt am Main (DE)

(72) Inventors: Helge Bode, Oberursel (DE); Claudia Buechel, Mainz (DE); Frederik Barka, Frankfurt (DE); Wolfram Lorenzen, Frankfurt am Main (DE)

(21) Appl. No.: 14/241,464

(22) PCT Filed: Sep. 6, 2012
(86) PCT No.: PCT/EP2012/067432

§ 371 Date: May 21, 2014

(30) Foreign Application Priority Data
Sep. 6, 2011 (DE) 102011113283.3

(51) Int. Cl. C12N 15/113; C11B 1/10; C12N 9/20

(52) U.S. Cl. CPC C12N 15/1137; C12N 9/20; C11B 1/10
USPC 536/23.1; 435/243; 435/198; 435/134; 554/1

(57) ABSTRACT

The present invention relates to a method of increasing the lipid content in an organism, in particular a micro algae by modulating the activity of a triacylglycerol (TAG) lipase. The invention also concerns organisms produced by this method, or microalgae, with increased lipid content, as well as their use in industry and medicine; in particular to obtain omega-3 unsaturated fatty acids. The invention relates furthermore to a nucleic acid from Phaeodactylum tricornutum, which encodes a new TAG lipase, and its use in influencing the proportion of fatty acids, in particular the content of polyunsaturated fatty acids in biomass.

The present invention relates to a method of increasing the lipid content in an organism, in particular a micro algae by modulating the activity of a triacylglycerol (TAG) lipase. The invention also concerns organisms produced by this method, or microalgae, with increased lipid content, as well as their use in industry and medicine; in particular to obtain omega-3 unsaturated fatty acids. The invention relates furthermore to a nucleic acid from Phaeodactylum tricornutum, which encodes a new TAG lipase, and its use in influencing the proportion of fatty acids, in particular the content of polyunsaturated fatty acids in biomass.

### DESCRIPTION

Microalgae produce large amounts of lipids, including a significant proportion of high polyunsaturated omega-3 fatty acids, which are essential for human nutrition and health. For this reason, micro-algae are of great biotechnological and industrial interest in two respects: one, they provide an alternative to fish oil as a source for the production of omega-3 fatty acids and may in the future be an alternative raw material for the production of biodiesel.

Currently the production of algae biomass is, compared to other sources of raw materials, not lucrative and far too expensive to compete with the established sources. High-quality omega-3 fatty acids are currently produced from fish oil, and biodiesel among other things is derived from palm oil. To date the only marketed production of omega-3 fatty acids from microalgae is the production of docosahexaenoic acid (DHA). Therefore, it is essential to increase the quantity of lipid in the algae to improve their competitive capacity.

Microalgae, as many other organisms as well, store chemical energy in the form of lipids. The fatty acids are stored mainly in the form of triacylglycerols (TAG), so to say as an energy store. As necessary, for example when the algae cannot meet their energy needs through photosynthesis or through the breakdown of other energy storage forms (e.g. starch), the storage-lipids are activated and introduced into the metabolism—therefore get degraded and their energy is retrieved. The initiating reaction of this activation is the same in all organisms. It is catalyzed by a specific class of enzymes, the triacylglycerol (TAG) lipases (EC 3.1.1.3).

Lipases catalyze the reaction of the cleavage of fatty acids from a lipid, such as TAG, under the consumption of water (lipolysis). During the degradation di- and monoacylglycerides are produced as intermediates, and finally free fatty acids and glycerol. Lipases are generally of great industrial importance and are applied in a variety of processes. Thus, lipases are used in fat-chemistry for the production of soaps, or are used for the treatment of foods, for example, the spreading properties of butter.

The diatom Phaeodactylum tricornutum produces, in addition to many other lipids, to a high degree the omega-3 fatty acid eicosapentaenoic acid (EPA, 20:5 n-3). The importance of EPA for human metabolism has been shown in numerous studies (among others Braden and Carol 1986; Simopoulus 1991; Ramjor et al. 1996) and is already used as nutritional supplement and for the treatment of inflammatory-rheumatic disorders in medicine (e.g. EPAMAX by Merck). These EPA-products are currently obtained solely from fish oil.

Lipases are known in the state of the art. The German publication document DE 100 26 845 describes the isolation of a nucleic acid from Arabidopsis that encodes a TAG lipase. It is proposed that the Arabidopsis TAG lipase is to be used to produce plants with altered lipid content. Thereby, DE 100 26 845 suggests to increase TAG lipase expression in transgenic plants by genetic constructs. However, this would result in a reduced content of TAG, and thereby of storage lipids.

The biotechnological production of lipid fractions containing EPA or DHA from microorganisms, in particular seeds of plants or marine organisms, is described in EP 1 392 623. The isolated lipid fractions are in particular to be used as a dietary supplement. The extraction of lipid fractions from marine microorganisms such as micro algae is likewise proposed.

Based on the prior art, it is therefore an object of the invention to provide new possibilities for the production of lipids and lipid fractions from biomass. In particular, the present invention seeks to overcome the problems of poor energy balance of the production of lipids from algae-biomass and thus to make industrial applicability accessible.

The above object is solved in a first aspect by a method for increasing the lipid content in an organism, comprising the steps of (a) providing an organism in which the lipid content is to be increased, (b) modulation of the expression and/or function a lipase in the organism, wherein the lipase is encoded by a nucleic acid sequence, comprising (i) a sequence selected from SEQ ID NO: 1 to 10, or comprising (ii) a sequence having at least 50% sequence identity to one of SEQ ID NOs: 1 to 10 or comprising (iii) a sequence which, under standard conditions, hybridizes to a sequence of SEQ ID NOs: 1 to 10.

For all embodiments of the present invention the term “lipase” shall also denote all isoforms of the lipase contained in the SEQ ID NO: 1. Particularly preferred are isoforms whose coding sequence is defined by the stop codon at position 3449 in SEQ ID No. 1. In particular, an isoform of the lipase is preferred, having a coding sequence located between the stop codon at position 3449 in SEQ ID NO: 1 and a start codon selected from the positions 663 (SEQ ID NO: 2), 969 (SEQ ID NO: 3), 977 (SEQ ID NO: 4), 1028 (SEQ ID NO: 5), 1055 (SEQ ID NO: 6), 1058 (SEQ ID NO: 7), 1178 (SEQ ID NO: 8) or 1218 (SEQ ID No.9) in SEQ ID No. 1. Particularly preferred is the isoform of the lipase, having the coding sequence located between the stop codon at position 3449 in SEQ ID NO: 1 and the start codon at position 663 (SEQ ID NO: 2).

In the context of the invention described herein it has been surprisingly found that it is advantageous to inhibit the expression of the key enzyme of the fat metabolism—the TAG lipase—in order to effect an increased lipid accumulation. The invention has been confirmed exemplary by the fact that a genetically engineered mutant of Phaeodactylum compared to the wild type accumulated significantly more lipids while showing comparable growth rates.

The suppression of a central enzyme of fatty acid catabolism results as shown herein into such an increase of the lipid content. Moreover, the principle of increasing the lipid content by suppressing the expression of the TAG lipase should be applicable in other species of algae; as long as the respective target gene can be identified.

Accordingly, the invention pertains in another embodiment to the above method, wherein the organism is preferably selected from the Chromalveolata, preferably the Bacillariophyceae (diatoms), preferably the Naviculales, preferably the Phaeodactylaceae, particularly preferably Phaeodactylum tricornutum; for example the organism is provided in the form an algal culture.

Furthermore, it is preferred that the lipase is encoded by a nucleic acid sequence, comprising a sequence which is identical to at least 50% to a sequence of SEQ NOs: 1 to 10. In this case, the lipase is in an embodiment characterized by its activity as TAG lipase. Additionally, it is preferred that the lipase is encoded by a nucleic acid comprising a sequence identical to a degree of 55%, 60%, 65%, 70%, 75%, 80%, 85%, 90%, 95%, 96%, 97%, 98%, 99% and 100% to one of the sequences of SEQ ID NOs: 1 to 10.

In addition, it is preferred that the lipase is encoded by a nucleic acid sequence which hybridizes under standard conditions to a nucleic acid of SEQ ID NOs 1 to 10. The term “standard conditions” means in particular in the context of nucleic acid hybridization, that the conditions are sufficiently stringent. Stringent conditions mean in particular a high temperature at low salt concentration. Stringent hybridization conditions are well known to those skilled in molecular biology: see, Sambrook et al, Molecular Cloning: A Laboratory Manual (1989) Cold Spring Harbor Laboratory Press, New York, USA.

The above-described nucleic acid sequences also encompass fragments, derivatives and allelic variants of the above described DNA sequences which code for a protein having the biological activity of a lipase, in particular a TAG lipase. By “fragments” parts of the nucleic acid sequence are meant that are long enough to encode one of the described proteins. Therefore, the nucleic acids of the present invention are preferably DNA or RNA molecules. The term “derivative” means in this context that the sequences differ from the DNA sequences described above at one or several positions, but still show a high degree of homology, i.e. sequence identity, to these sequences. The deviations from the nucleic acid sequences described above could have been formed for example by deletion, substitution, insertion or recombination.

The above described variants or derivatives of lipases are enzymes that are altered in their sequence. However, the novel enzyme variants retain their original biological and catalytic activity. The sequence changes may be naturally occurring sequence variation that develop, for example, due to the degeneracy of the genetic code, or are introduced artificially, for example by targeted mutagenesis of the respective sequences. Such techniques are well known to the skilled person.

The present method intends to make an increase of the content of lipids in an organism possible. As the lipase acts as a central point in the catabolism of lipids, it is preferred in context of the present invention that the modulation of the expression and/or function of the lipase is a reduction of the expression and/or function. In particular it is preferred that the expression of the lipase is reduced.

In a further embodiment of the invention, a method is described in which the lipid content in the organism is increased to 110% to 250% compared to an untreated control; preferably wherein the lipid content is increased to 120% to 200%, to 150%) to 200%, more preferably to 180% to 200%, and most preferably increased to about 190%. In other words, the present method causes an increase in the absolute lipid content of the mutant compared to the wild-type by a factor of at least 1.1, more preferably 1.2, 1.3, 1.4, 1.5, 1.6, 1, 7, 1.8, 1.9 or 2 or even beyond.

In this regard in one embodiment also individual fatty acids may be increased in the organism. Here, the present method enables an increase of the content of fatty acids having 14, 16, 18, 20, 22 or more carbon atoms which have 1, 2, 3, 4, 5, or more double bonds. In particular, the content of 14:0, 16:4, 16:3, 16:1, 18:4, 18:2, 18:1, 18:0, 20:4, 20:5 and 22:6 fatty acids (carbon atoms:double bonds) is increased.

The inventive method provides an increase in the lipid content in organisms, wherein the increase of the lipid content surprisingly takes place at a constant growth rate of the organism.

More preferred is in one embodiment of the invention a method wherein the change in expression and/or function of a lipase in the organism is attained by inhibiting the expression and/or function of the lipase. In this respect it is preferred that the inhibition of the lipase is achieved by introducing and expressing of an RNA interference construct in the organism. The RNA interference construct is directed against the sequence of the lipase.

By means of expressing a so-called antisense RNA (an RNA complementary to the target mRNA) the expression of the corresponding target gene can be suppressed in micro-algae (Riso De et al. 2009). The expression of an antisense RNA complementary to the mRNA of the TAG lipase in Phaeodactylum inhibits the expression of the TAG lipase, whereby the mutant can activate or degrade the stored lipids to a lesser extent. In fact, it was shown as part of the invention described herein, that the respective genetically modified Phaeodactylum-mutant according to first results has significantly higher lipid content and a significant accumulation of triacylglycerol, at comparable growth rates.

Therefore, the method pertains in a further embodiment to the use of RNA interference technology (RNAi) for the modulation of the lipase activity, wherein the RNA interference construct is selected from a sense construct, an antisense construct or an inverted repeat construct. An “inverted repeat” is a repeating nucleic acid sequence that is arranged in reverse—thus inverted—orientation. An inverted repeat can thus hybridize with itself whereby the molecule folds as a so-called hairpin structure. Therefore, the expression of an inverted repeat leads to a hairpin RNA, and thereby yields a double-stranded RNA that triggers the RNAi process in the cell. The design and the expression of such constructs are well known and do not pose any special challenges for the artisan.

RNAi constructs are characterized in that they have an at least highly homologous sequence to the mRNA to be inhibited. This is due to the fact that the cellular RNAi machinery is aligned in accordance to the sequence similarity of the RNAi-construct to the target mRNA. During RNAi expression is post-transcriptionally inhibited. This occurs for example, by degradation of the target mRNA by a nuclease, or by inhibiting the translation of the target mRNA. Thus, in a further preferred embodiment the method pertains to an antisense construct that comprises a sequence that is identical to at least 50% to a sequence selected from SEQ ID NOs: 1 to 10, or that can hybridize with such a sequence under standard conditions.

In this case, an anti-sense construct is particularly preferred that has the ability to inhibit a lipase, wherein the lipase is encoded by a nucleic acid sequence comprising (i) a sequence selected from SEQ ID NOs: 1 to 10, or comprising (ii) a sequence having at least 50% sequence identity to one of SEQ ID NOs: 1 to 10, or comprising (iii) a sequence which, under standard conditions hybridizes to a sequence of SEQ ID NOs: 1 to 10. Particularly preferred is a lipase, which is encoded by a nucleic acid sequence which comprises SEQ ID NO: 10, or is identical to at least 50% to SEQ ID NO: 10.

Furthermore it is well known to those skilled in the art that RNAi constructs are not only directed solely to the coding region of a gene, but also to non-translated regions, in particular in the 5′ and 3′ region of the mRNA of a target gene. Accordingly, it is preferred in one embodiment that the RNAi-construct comprises a sequence that is located in the 5′ region of one of the initiation codons selected from the positions 663, 969, 977, 1028, 1055, 1058, 1178 or 1218 in SEQ ID NO: 1, or located in the 3′ region of the stop codon at position 3449.

Particularly preferred is that the antisense construct comprises the SEQ ID NO: 10.

Preferably, the antisense construct, sense construct, or inverted-repeat construct is introduced to transform a cell of the organism. Therefore, the organism is preferably a microorganism such as a microalga. Here, all known possibilities to perform the transformation process in the art can be used to perform the method. In the case of a transformation of microalgae a biolistic transformation is preferred, as exemplary described in the examples.

To ensure the expression of the inhibitory construct in the organism, the construct contains in a preferred embodiment an expression promoter. Promoters allow for a constitutive or inducible expression of the inhibitory sequence, for example, of the antisense construct, of the sense construct or of the inverted repeat. For this purpose, the expression promoter is operably linked with the inhibitory construct. Particularly preferred for the expression of antisense constructs in microalgae is the use of the nitrate reductase promoter “ProNr” which depending on the culture conditions allows for an induction or constitutive expression of the construct.

The present method is not limited only to the application of RNAi. Rather, all processes are possible and encompassed, that lead to a reduction in the activity of the herein disclosed lipase. This can be achieved also by the introduction of mutations into the genomic sequence of the organism. Such mutations can affect either the enzyme in its activity or stability, or lead to a reduction in the expression of the lipase. To reduce the expression of the lipase, the mutations are preferably introduced into the regulatory sequences of the respective gene. For this purpose, particular promoter or enhancer sequences of the gene are desirable. Techniques of mutagenesis include, for example, the introduction of point mutations or deletions by homologous recombination; or any other technique that results in the destruction of the open reading frame of the lipase. Furthermore, methods are conceivable, which cause specific inhibition of the catalytic activity of the lipase via inhibitory molecules such as so-called “small molecules” or anti bodies directed at the lipase.

In this context, the present invention comprises, in another aspect, a method for identifying modulators of a lipase, wherein the lipase is encoded by a nucleic acid sequence comprising (i) a sequence selected from SEQ ID NOs: 1 to 10, or comprising (ii) a sequence having at least 50% sequence identity to one of SEQ ID NOs: 1 to 10, or comprising (iii) a sequence which under standard conditions hybridizes to a sequence of SEQ ID NOs: 1 to 10. The method comprises the steps of providing the lipase, bringing the lipase into contact with a potential modulator, and a determination of the catalytic activity of the lipase in the presence of the candidate modulator.

Preferred modulators may in particular be selected from known collections of so-called “small molecules”, or be nucleic acids and proteins, specifically inhibitory antibodies. In this case, antagonists of the activity of the lipase are particularly preferable as modulators in context of the present invention.

The activity of lipase in the above process can be tested by determining the conversion of TAG to diacylglyceride and free fatty acids, mono acylglyceride and free fatty acids and/or glycerol and free fatty acids.

Another aspect of the present invention is directed to a method for producing an organism with increased lipid content, comprising performing the above described method.

One aspect of the invention relates to an organism that has been prepared by the process described above. In this context, “production” means the genetic modification of the organism that leads to an increase in lipid content as described above. This concerns in particular microalgae, which exhibit increased lipid content by means of genetic modification via the introduction of antisense constructs.

In a further aspect, the present invention is directed to a method for improving the cultivation characteristics of an organism, comprising the modification of expression and/or function of a lipase in the organism, wherein the lipase is encoded by a nucleic acid sequence comprising (i) a sequence selected from SEQ ID NOs: 1 to 10, or comprising (ii) a sequence having at least 50% sequence identity to one of SEQ ID NOs: 1 to 10, or comprising (iii) a sequence which, under standard conditions hybridizes to a sequence of SEQ ID NOs. 1 to 10.

In course of the modulation of the expression of the lipase described herein, it has also been surprisingly found that microalgae which are impaired in the expression of the lipase according to the herein disclosed sequence SEQ ID NO:1 display particularly advantageous cultivation characteristics. The modified micro algae show compared to the wild type variant a reduced deposition of algae-remains on the flask wall of an algae culture. Especially for cultivation in large reactors such deposits are problematic, since a portion of the biomass becomes inactive, and the reactors must also be cleaned in short intervals.

Accordingly, it is a preferred embodiment of the method, when the organism is selected from the Chromalveolata, preferably the Bacillariophyceae (diatoms), preferably the Naviculales, preferably the Phaeodactylaceae, particularly preferably Phaeodactylum tricornutum.

In a further embodiment of the method the improving of the cultivation-characteristics is characterized by the reduction of the deposits of the algae in a culture container.

In a further aspect of the invention described herein, a nucleic acid is provided, comprising (i) a sequence selected from SEQ ID NOs: 1 to 10, or comprising (ii) a sequence having at least 50% sequence identity to one of SEQ ID NOs: 1 to 10, or comprising (iii) a sequence which, under standard conditions hybridizes to a sequence of SEQ ID NOs: 1 to 10. The respective preferred embodiments as described above are applicable to the claimed sequences. In particular, the invention is concerned with sequence variants of the new lipase.

Another aspect of the invention resides in a vector comprising one of the nucleic acids described herein. In this context, expression vectors are particularly preferred which contain all the necessary sequences known in the art that allow for the expression of a nucleic acid. The invention therefore furthermore relates to a host cell comprising a nucleic acid of the present invention or a vector of the invention.

Another aspect of the invention is a lipase having an amino acid sequence encoded by the nucleic acids of the invention.

Another aspect of the invention is directed to a method for extraction of lipids comprising: (a) providing a culture of organisms having an increased lipid content in accordance with the present invention, (b) expanding the culture until a desired culture density is reached, and (c) extraction of the lipids from the culture.

Thus, in a further aspect, also an alga-extract or a lipid mixture is comprised that was prepared by the method for the production of lipids as described above.

The methods and materials described herein can be used in the manufacture of food supplements, as well as in the cosmetic and pharmaceutical fields

The present invention will be described in greater detail in the following by way of examples with reference to the accompanying drawings, without limiting the invention thereby.

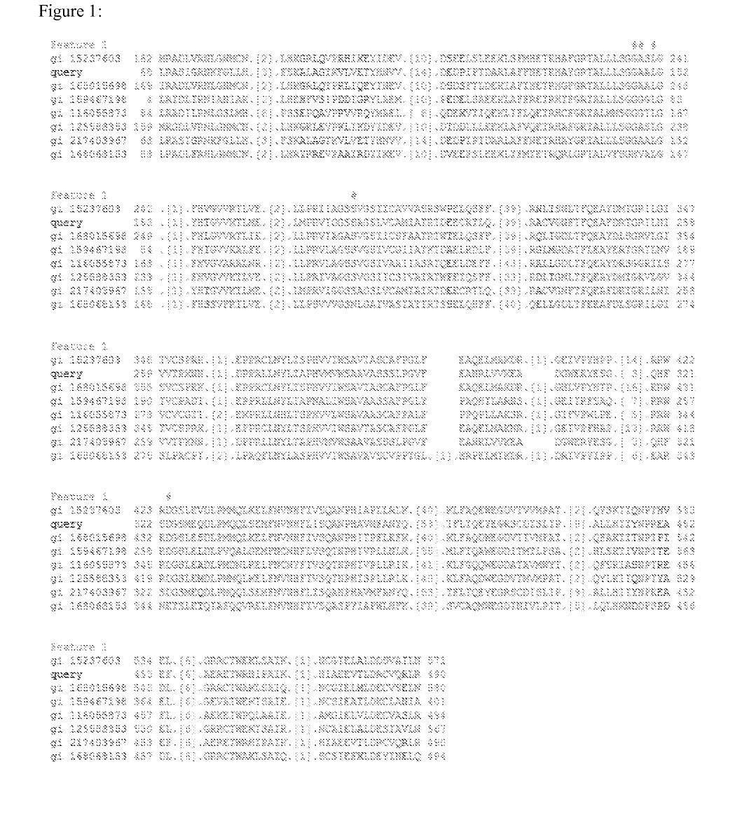

FIG. 1: Automatic BLAST result with other amino acid sequences of other Databases of NCBI. The characteristic amino acids of the active site “active site” are marked with (#). (Http://www.ncbi.nlm.nih.gov/Structure/cdd/cddsrv.cgi)query/gi=217403967 part of the TAG lipase Phaeodactylum (SEQ ID NO 1.), gi=15237603 SDP1 (SUGAR-DEPENDENT1), triacylglycerol lipase [Arabidopsis thaliana]; gi=168015698 predicted protein [Physcomitrella patens subsp. patens]; gi 159467198=predicted protein [Chlamydomonas reinhardtii]; gi 116055873=Predicted esterase of the alpha-beta hydrolase superfamily (ISS) [Ostreococcus tauri]; gi 125588353=hypothetical protein OsJ_{—}13065 [Oryza sativa Japonica Group]; gi 168068153=predicted protein [Physcomitrella patens subsp. patens]

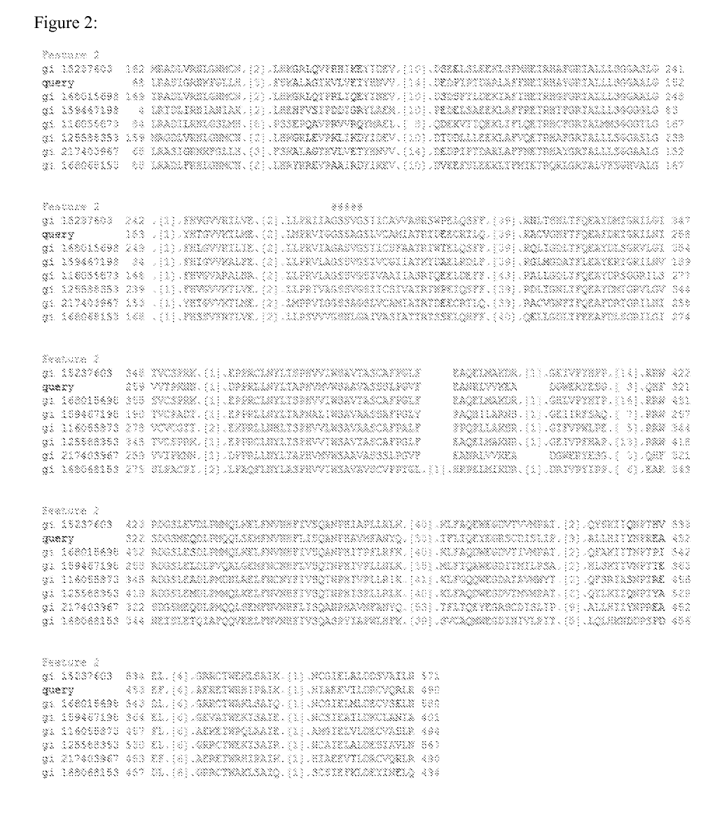

FIG. 2: Automatic BLAST result with other amino acid sequences of other databases of NCBI. The characteristic amino acids of the feature “nucleophilic elbow” are marked with (#). (Http://www.ncbi.nlm.nih.gov/Structure/cdd/cddsrv.cgi)query/gi=217403967 part of the TAG lipase Phaeodactylum (SEQ ID NO: 1); gi=15237603 SDP1 (SUGAR-DEPENDENT1); triacylglycerol lipase [Arabidopsis thaliana]; gi=168015698 predicted protein [Physcomitrella patens subsp patens.]; gi=159467198 predicted protein [Chlamydomonas reinhardtii]; gi 116055873=Predicted esterase of the alpha-beta hydrolase superfamily (ISS) [Ostreococcus tauri]; gi 125588353=hypothetical protein OsJ_{—}13065 [Oryza sativa Japonica Group], gi 168068153=predicted protein-latest] [Physcomitrella patens subsp patens.]

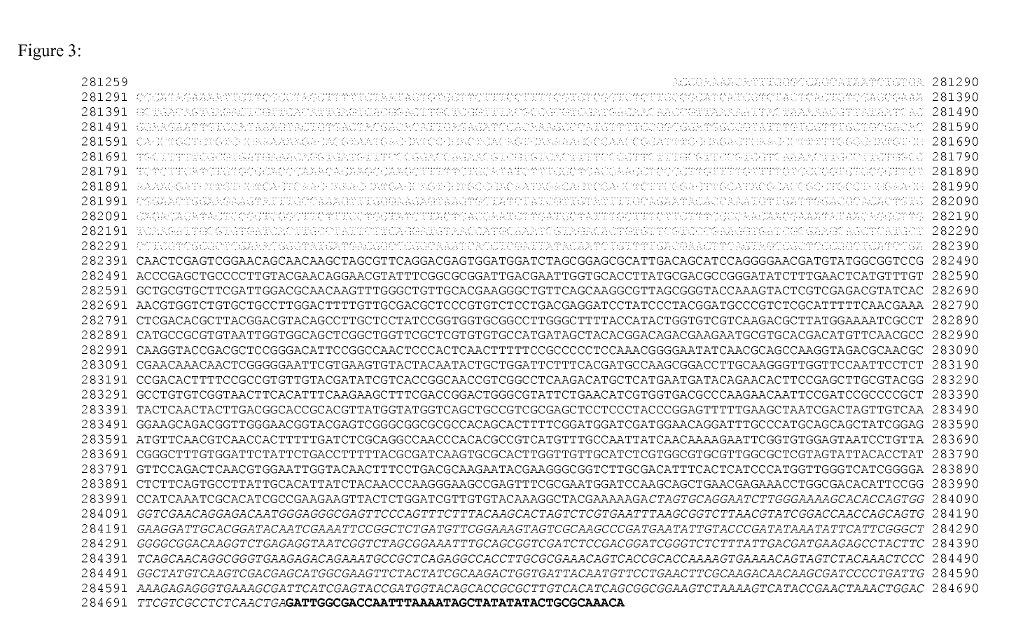

FIG. 3: the gene sequence of the lipase of SEQ ID Chr_{—}24:281259-284752 (SEQ ID NO: 1)

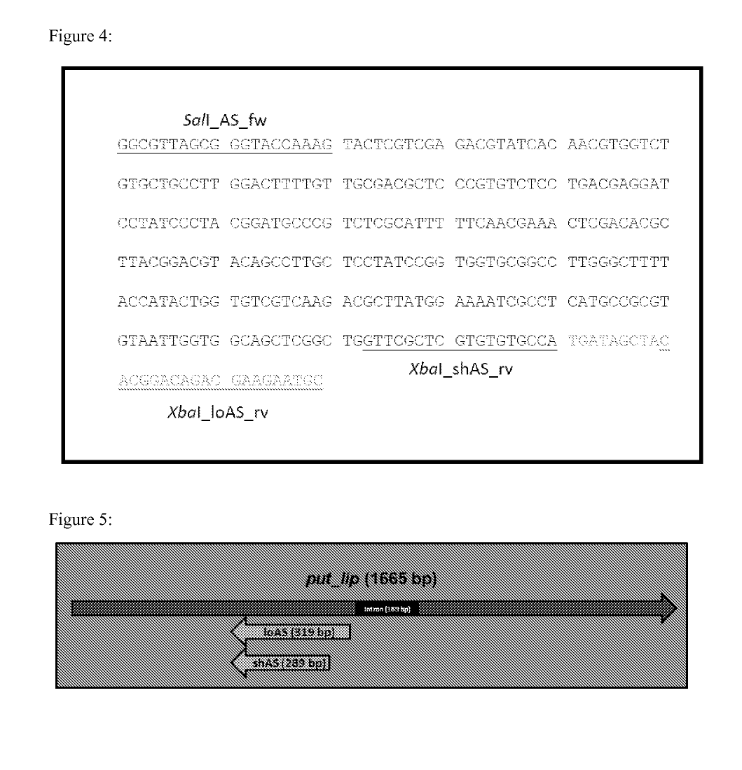

FIG. 4: trigger sequence for the antisense constructs IoAS (SEQ ID NO: 11) and shAS (SEQ ID NO: 10). The primer binding sites are underlined.

FIG. 5: schematic illustration of the annotated gene sequence of the lipase gene (put_lip) and the antisense constructs shAS and IoAS

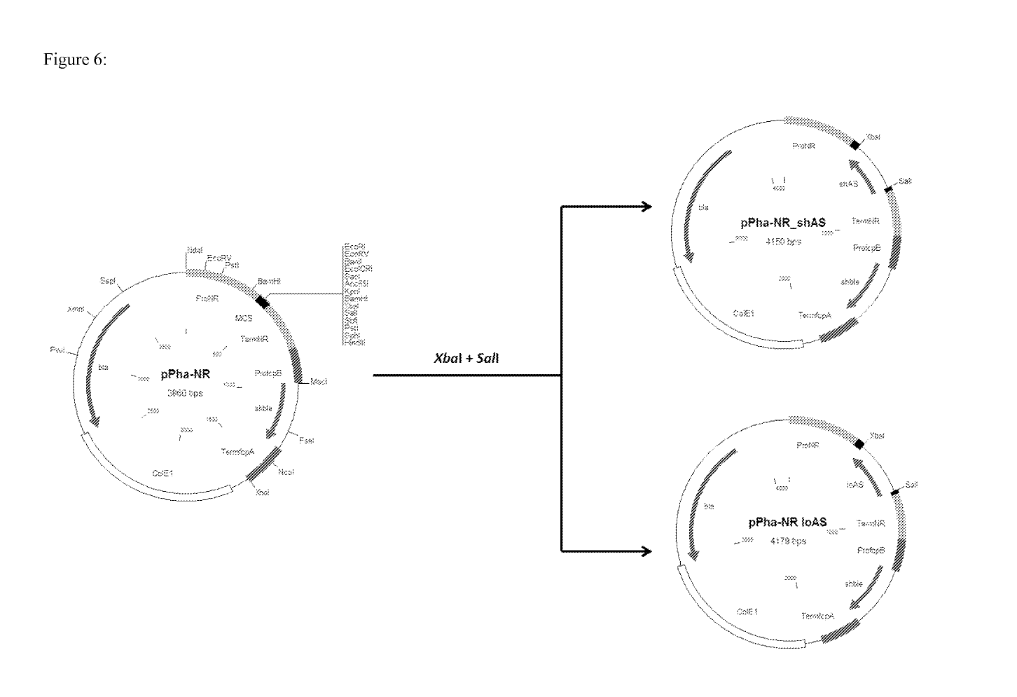

FIG. 6: overview of the cloning of the antisense fragments into the vector pPhaNR

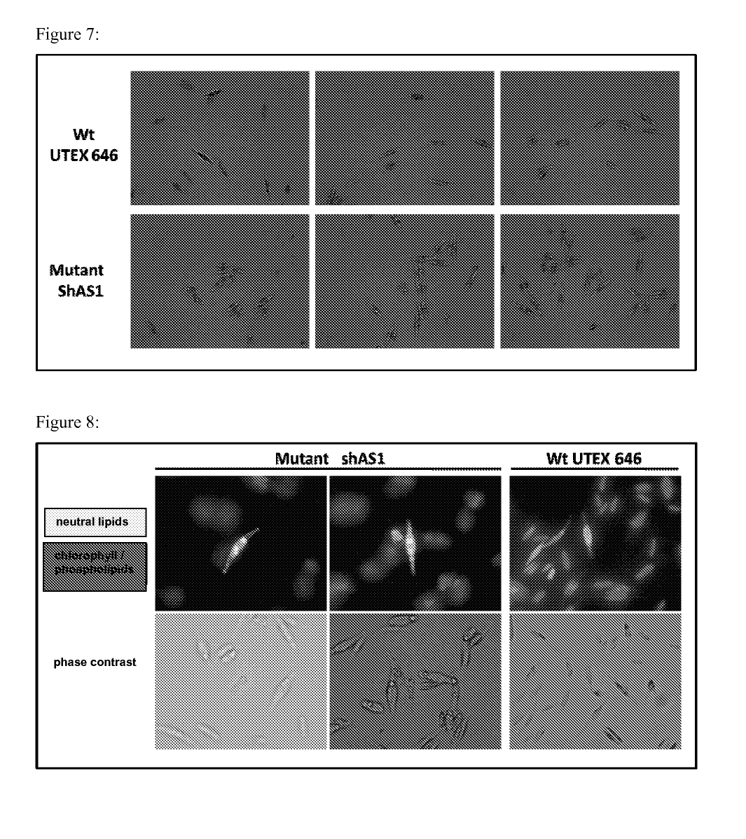

FIG. 7: optical microscopic pictures of P. tricornutum wild type UTEX 646 and the mutant ShAS1.

FIG. 8: fluorescence microscopic pictures of P. tricornutum wild type UTEX 646 and the mutant ShAS1. The cells were in advance treated with the fluorescent dye nile red, to make the lipid droplets visible within cells.

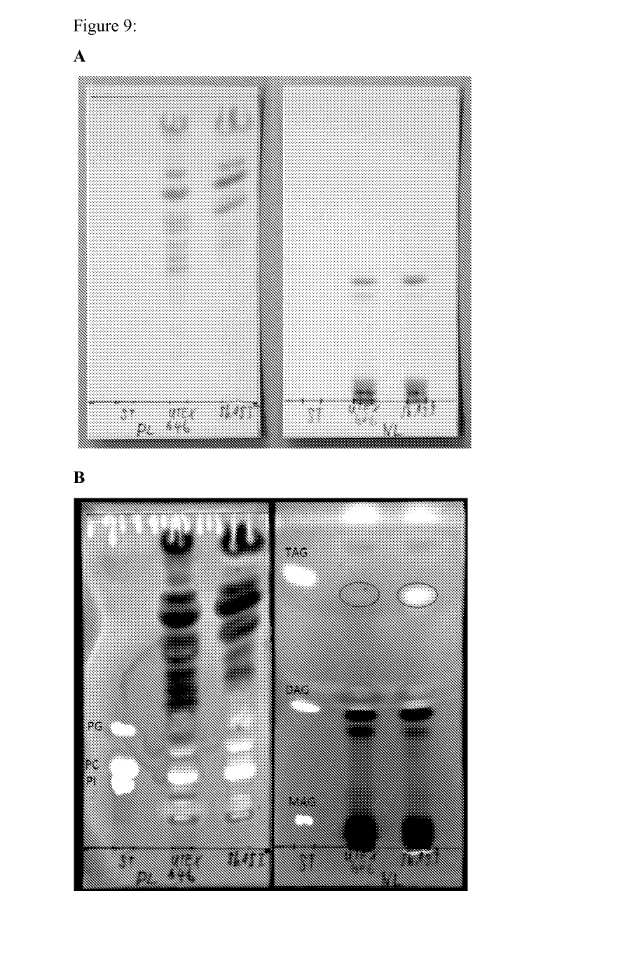

FIG. 9: thin layer chromatography of the lipid extracts of UTEX 646 and Mutant ShAS1. (Stationary phase: silica gel 60 F254, Merck). Mobile phases are NL: hexane-diethyl ether-acetic acid (80:20:1), and PL: chloroform-methanol-water (95:35:5). A: chromatography in visible light. B: primolin dye. In the standard the following mean PG: phosphatidylglycerol, PC: phosphatidylcholine, PI: phosphatidylinositol, TAG: triacylglycerol, DAG: diacylglycerol, MAG: monoacylglycerol.

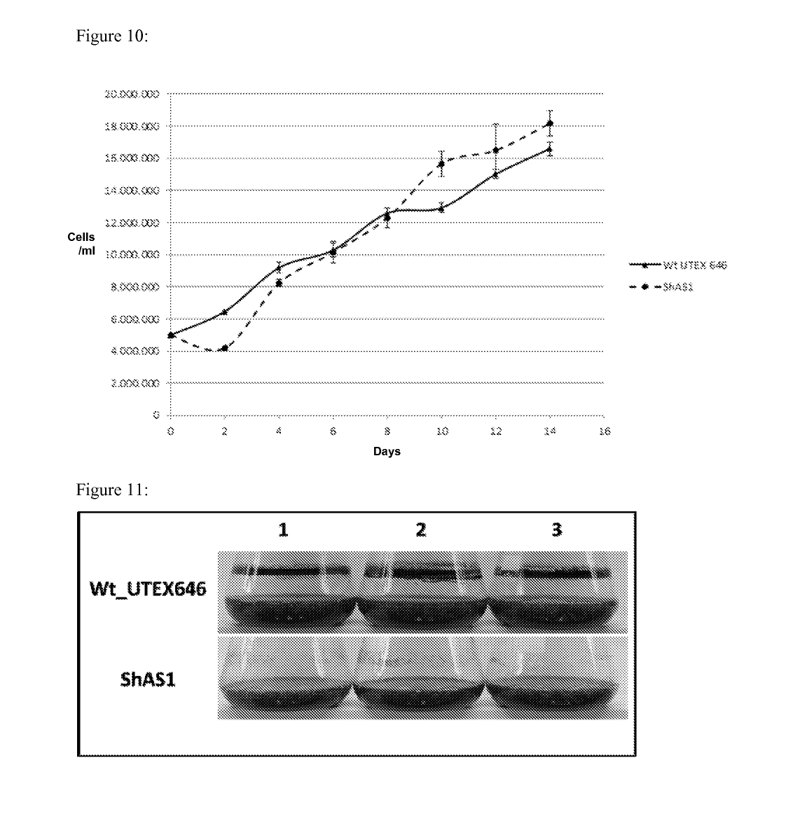

FIG. 10: growth curves of the wild type (Wt UTEX 646), and the antisense mutant (ShAS1)

FIG. 11: culture flasks of the wild type UTEX 646 and the antisense mutant Shas1

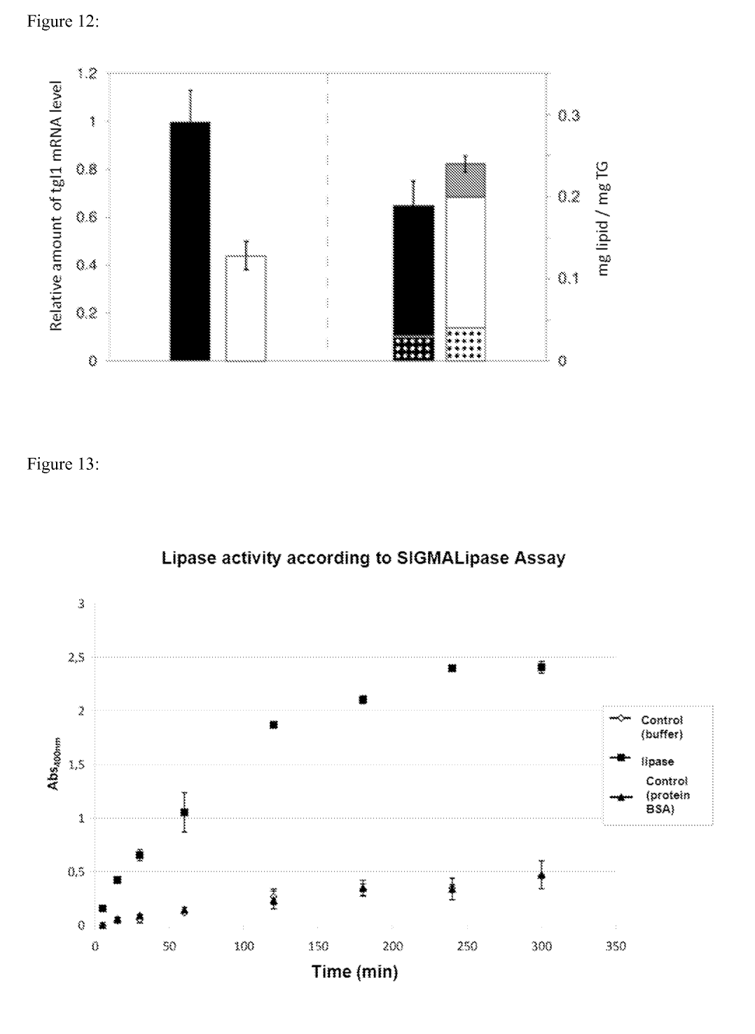

FIG. 12: left side: relative amount illustration of qRT-PCR. Three different RNA isolations were used to determine the relative expression levels of the new lipase (SEQ ID No 2.) in wild-type (Wt, black bars) or mutant (ShAS1, white bars), and compared with histone H4 as reference. Quantification of mRNA levels was carried out in triplicates. The average value for wild-type was used as a calibrator and set to 1. Right side: lipid yield per dry weight (TG) from four independent cultures of P. tricornutum wild-type (black bars) and lipase knock-down mutant (ShAS1, white bars). The pigment content is dotted and TAG (gray) is displayed.

FIG. 13: lipase activity assay according to SIGMALipase assay. The lipase in question is the new lipase according to the invention (encoded by SEQ ID NO. 2), which was recombinantly overexpressed in E. coli.

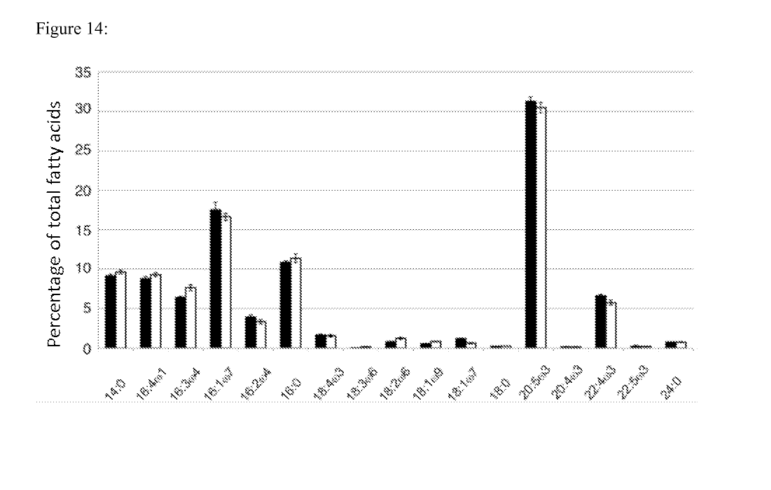

FIG. 14: relative proportion of fatty acids measured as methyl esters (FAME) in lipid extracts from P. tricorntum wild-type (dark bars) and in lipase knock-down mutant (light bars). The data represent the average of three independent experiments.

### EXAMPLES

### 1. Description of the Lipase

### 1.1 Currently Annotated Gene Sequence of the New TAG Lipase

Putative TAG lipases were sought in the fully sequenced genome of Phaeodactylum tricornutum based on similarities with other TAG lipases. The part of the gene sequence of the potential TAG-lipase put lip from P. tricornutum containing the characteristic of TAG lipase sequences, is found among others in the databases from NCBI (National Center for Biotechnology Information). This partial sequence has the NCBI “Accession Number” XP 002184517.1 and is annotated as “hypothetical protein “PHATRDRAFT_{—}1971”. 1971 is the protein/gene ID of the gene data from the JGI (Joint Genome Institute, http://genome.jgi-psf.org/Phatr2/Phatr2.home.html). “CCAP 1055/1” describes the Phaeodactylum strain used for genome sequencing.

The gene sequence of the lipase including the coordinates of the individual bases is shown in FIG. 3.

### 1.2 Homology to Lipases of Other Organisms

Based on sequence homologies in the amino acid sequence of the corresponding protein to the nucleic acid sequence described here, the potential lipase is assigned to the group of “patatin-SDP1-like” lipases. In particular, two conserved domains can be found in the sequence: a catalytic center (“active site”), which matches with other lipases, as well as a second conserved domain “Nucleophilic Elbow”. In FIG. 1 the catalytic amino acids of the catalytic center and in FIG. 2 the characteristic amino acids of the “nucleophilic elbow” in the corresponding sequence are indicated by #.

A self-performed sequence comparison of the amino acid sequences of selected (potential) TAG lipases from other organisms, which are also assigned to the group of “Patatin-SDP1-like” lipases, shows the quality of the sequence homologies within this group (Table 1).

TABLE 1
Result of the alignment of the amino acid sequences of various
“patatin SDP1-like” lipases of other organisms; Phaeo = part of the TAG
lipase from P. tricornutum SEQ ID No. 1 (PHATRDRAFT_1971);
Taps = pot TAG lipase from T. pseudonana; Saccha = TAG lipase (Tgl3)
from S. cerevisiae; Arab = SDP1 from A. thaliana,
Chlamy = pot TAG lipase from C. reinhardtii;
homo = Human adipose TAG lipase

| SeqA [symbol] | Name [symbol] | Length [symbol] | SeqB [symbol] | Name [symbol] | Length [symbol] | Score [symbol] |
|---|---|---|---|---|---|---|
| 1 | Phaeo | 492 | 2 | Taps | 449 | 58.0 |
| 1 | Phaeo | 492 | 3 | Saccha | 642 | 22.0 |
| 1 | Phaeo | 492 | 4 | Arab | 825 | 30.0 |
| 1 | Phaeo | 492 | 5 | Chlamy | 440 | 30.0 |
| 1 | Phaeo | 492 | 6 | Homo | 504 | 4.0 |
| 2 | Taps | 449 | 3 | Saccha | 642 | 21.0 |
| 2 | Taps | 449 | 4 | Arab | 825 | 29.0 |
| 2 | Taps | 449 | 5 | Chlamy | 440 | 25.0 |
| 2 | Taps | 449 | 6 | Homo | 504 | 4.0 |
| 3 | Saccha | 642 | 4 | Arab | 825 | 18.0 |
| 3 | Saccha | 642 | 5 | Chlamy | 440 | 22.0 |
| 3 | Saccha | 642 | 6 | Homo | 504 | 7.0 |
| 4 | Arab | 825 | 5 | Chlamy | 440 | 43.0 |
| 4 | Arab | 825 | 6 | Homo | 504 | 9.0 |
| 5 | Chlamy | 440 | 6 | Homo | 504 | 8.0 |

### 2. Description of the Inhibition of Lipase

A targeted gene “knock-out” for example by homologous recombination is currently not possible in Phaeodyctylum and other diatoms. However, the inhibition of gene expression as a “knock-down” by the recombinant expression of antisense constructs is considered an established method (De Riso et al. 2009). This was used for the herein described mutants. Alternatively, also inverted-repeat constructs may be used (De Riso et al.2009).

### 2.1 Construct Production

For the inhibition of the potential TAG lipase two constructs were cloned, that express
1. a 290-bp antisense RNA (short antisense; shAs—SEQ ID NO: 10) and
2. a 319-bp antisense RNA (long antisense; IoAs—SEQ ID NO: 11)
   against the lipase gene put lip (SEQ ID NO: 1). For the amplification of the antisense constructs from genomic DNA of Phaeodyctylum the following primers were used:

SalI_AS_fw:
(SEQ ID NO: 12)
5′-CTTC GTC GAC GGC GTT AGC GGG TAC CAA AG-3′
XbaI_shAS_rv:
(SEQ ID NO: 13)
5′-CTT TCT AGA TGG CAC ACA CGA GCG AAC-3′
XbaI_loAS_rv:
(SEQ ID NO: 14)
5′-CTT TCT AGA GCA TTC TTC GTC TGT CCG TG-3′

The primers each contain at the 5′ end in addition to the lipase-specific sequence restriction sites for subsequent cloning into the expression vector pPha-NR (indicated as underlined in the primer sequences). The additional bases at the 5′ end are required for efficient restriction of the PCR products.

The trigger sequence for the antisense constructs IoAS (SEQ ID NO: 11) and shAS (SEQ ID NO: 10) is shown in FIG. 4. The primer binding sites are underlined in the sequence. A schematic representation of the location of the antisense constructs in the lipase sequence is shown in FIG. 5.

The antisense fragments were cloned via SalI/XbaI recognition sites into the vector pPha-NR (FIG. 6) and E. coli was transformed with the vector. After extraction of the plasmids from E. coli, the DNA thus obtained was used for transformation of P. tricornutum (see below).

Recombinant expression of the antisense constructs in the mutants is therefore under the control of the promoter “ProNR”. This is the promoter of nitrate reductase from P. tricornutum. This promoter is usually inducible, that is, there is the possibility that expression of the antisense constructs is turned on or off, respectively. Under our chosen growth conditions of P. tricornutum the nitrate reductase promoter is however constitutively active.

2.2 Transformation of Phaeodactylum tricornutum:

For the transformation of the wild type, first a culture was harvested and concentrated in the early exponential phase of growth (approximately 4-5 days old and approximately 3-5*10⁶ cells/ml). Subsequently, the cells were transformed biolistic. For this approximately 1 μg DNA was precipitated on 3 mg washed tungsten particles with the help of spermidine and CaCl2, washed with ethanol and the particles so prepared were used for bombardment of cells on agar plates in growth medium (10⁸ cells per plate). The next day cells were transferred to agar plates containing zeocin and placed in liquid culture after another 3 weeks.

### 3. Results

### 3.1 Characterization of the Lipase Mutant ShAS1

The mutant ShAS1, which was noticed due to a strikingly high number of lipid droplets within the cells at a routine microscopic cell counting, was thereafter extensively studied (FIGS. 7 and 8). qRT-PCR was performed to determine in cell culture the relative mRNA levels of the wild-type (WT) lipase and RNAi knockdown mutant (ShAS1). The amount of lipase transcripts was normalized in both cases by the constitutively expressed histone H4 mRNA. The average value for wild-type lipase mRNA was set to 1. The relative expression of the lipase in the mutant was reduced to 44% (+/−6%) by the RNAi construct. These data are based on the averages of three independent RNA isolations (FIG. 12, left side).

### 3.2 Lipid Extraction of Wildtype UTEX 646 and Mutant ShAS1:

For the lipid extraction and analysis 5 liter of each airlift cultures of Phaeodactylum wildtype UTEX 646 and lipase “Knock-down” mutant shAS1 were cultured. The flasks were inoculated with approximately 500,000 cells/ml and cultured for 4 days in ASP medium under the following conditions: 18° C., light-dark cycle: 16 hours/8 hours; approximately 40 μE m⁻² s⁻¹. Cell number at harvest: 3-4×10⁶ cells/ml (→Exponential growth phase).

### Lipid Analysis:

Starting material for lipid extraction (cell counting after harvesting and parallel dry weight determination):

(In each case, 3 ml were used for lipid extraction)
UTEX646: 2.8×10⁹ cells/ml
ShAS1: 2.5×10⁹ cells/ml

This resulted in the following amounts of extract:
UTEX646: 56.2 mg (approximately 6.7 pg/cell according 0.22+/−0.03 mg lipid extract/mg dry weight
ShAS1: 96.5 mg (approximately 12.9 pg/cell according 0.28+/−0.01 mg lipid extract/mg dry weight of the mutant (FIG. 12, right side))

The amounts of extract were determined gravimetrically and contained not only the lipids but various pigments. Assuming, however, a similar pigment content in wild-type and mutant (FIG. 12, right side), the following ratio of lipid levels between wild-type and mutant can be calculated:
12.9 pg/cell (mutant) compared to 6.7 pg/cell (wild type)=1.93

This results in approximately 90% more extract per cell in the mutant compared to the wild type. In the mutant, there were 0.04±0.01 mg TAG/mg dry weight, but no TAG under this culture condition in the WT.

### 3.3 Thin-Layer Chromatography of the Lipid Extracts

The extracts were analyzed on thin-layer plates against standards (FIG. 9) and a strong enrichment of triacylglycerides in the mutant compared to the wild type (WT) was observed. In addition, a mass spectrometric investigation of the total fatty acid composition of wild-type UTEX 646 and mutant shAS1 after esterification (fatty acid methyl ester (FAME) analysis by GC-MS) (methyl acetate=ME) was performed (Tables 2 and 3, and FIG. 14).

TABLE 2
Fatty acid composition by FAME-GC-MS analysis

| | | | | Ratio of the absolute |
|---|---|---|---|---|
| | | | | amounts of |
| | | | Δ (Wt vs | each fatty acid (*) |
| Fatty acid | ShAS1 | UTEX646 | Mutante) | Δ (Wt vs Mutant) |
| 14:0 ME | 5.50% | 5.56% | −0.06% | +90% |
| 16:4w1 ME | 4.99% | 6.46% | −1.47% | +49% |
| 16:3w4 ME | 7.12% | 7.04% | 0.07% | +95% |
| 16:1w7c ME | 25.26% | 16.29% | 8.97% | +199% |
| 16:2w4.6 ME | 1.17% | 2.92% | −1.75% | −23% |
| 16:0 ME | 19.13% | 15.77% | 3.36% | +134% |
| 18:4w3 ME | 1.35% | 0.95% | 0.40% | +174% |
| 18:3w6.9.12 ME | n.d. | n.d. | — | — |
| 18:2w6.9 ME | 0.73% | 0.73% | 0.00% | +93% |
| 18:1w9c ME | 3.12% | 3.50% | −0.38% | +72% |
| 18:1w7c ME | 0.87% | 0.44% | 0.43% | +281% |
| 18:0 ME | 4.01% | 5.25% | −1.24% | +47% |
| 20:5w3.6.9.12.15 | 24.10% | 31.84% | −7.74% | +46% |
| ME | | | | |
| 20:4w3.6.9.12 | 0.08% | 0.09% | −0.01% | +71% |
| ME | | | | |
| 20:4w6.9.12.15 | n.d. | n.d. | — | — |
| ME | | | | |
| 22:6w3 ME | 2.24% | 2.51% | −0.27% | +72% |
| 22:5w3 ME | 0.10% | 0.19% | −0.09% | +1% |
| 24:0 ME | 0.23% | 0.45% | −0.22% | −2% |

(*) Based on the total amount or the lipid extract and FAME-GC-MS analysis (6.7 pg/cell for UTEX 646 and 12.9 pg/cell for UTEX 646 and 12.9 pg/cell for ShASl)

TABLE 3
Fatty acid composition classified according
to the acyl chain length of the fatty acids

| | ShAS1 | UTEX646 | Δ (Wt vs Mutant) |
|---|---|---|---|
| 14 series | 5.50% | 5.56% | −0.06% |
| 16 series | 57.67% | 48.49% | 9.18% |
| 18 series | 10.07% | 10.87% | −0.79% |
| 20 series | 24.18% | 31.93% | −7.76% |
| 22 series | 2.34% | 2.70% | −0.36% |
| 24 series | 0.33% | 0.64% | −0.30% |

### 3.4 Growth Comparison

For the comparison of growth, 3 independent culture flasks of 300 ml ASP medium were inoculated with 5×10⁶ cells/ml for wild-type and mutant. Cultures were incubated at a temperature of 18° C., light-dark cycle: 16 hours/8 hours; approximately 40 μE m⁻² s⁻¹ cultured as a batch culture. The cell number was determined using a Thoma counting chamber. The growth rates of the wild type UTEX 646 and the mutant ShAS1 showed no significant differences (FIG. 10).

### 3.5 Lipase Activity

The enzymatic activity of the isolated lipase of the present invention was examined further by the SIGMA lipase assays. To do this, the lipase was recombinantly overexpressed in E. coli. The new lipase of the invention showed already after a few minutes enzymatic activity compared with the controls with buffer or BSA protein (FIG. 13).

### 4. Additional Results

During the recording of the growth curve of the wild type UTEX 646 and the mutant ShAS1 the mutant showed a further remarkable property: usually, Phaeodactylum cells tend to settle on the edge of the culture flask (“Wall Growth”). This phenomenon, especially in the mass cultivation of microalgae is a big problem, since the photo bioreactors used must be laboriously cleaned of debris. Compared to the wild type, the mutant ShAS1 showed significantly reduced deposits on the culture flask wall (FIG. 11).

1. A method for increasing the lipid content in an organism, comprising the steps of
a. providing an organism, in which the lipid content is to be increased,
b. modulation of the expression function of a lipase in the organism, wherein the lipase is encoded by a nucleic acid sequence, comprising
i. a sequence selected from SEQ ID NOs: 1 to 10, or comprising
ii. a sequence of at least 50% sequence identity to one of SEQ ID NOs: 1 to 10, or comprising
iii. a sequence which hybridizes under standard conditions with a sequence of SEQ ID NOS: 1 to 10.

2. The method according to claim 1, wherein the organism is selected from the Chromalveolata.

3. The method according to claim 1, wherein the modulation of the expression and/or function is a reduction of the expression.

4. The method according to claim 1, wherein the lipid content is increased in the organism to 110% to 250% compared to an untreated control.

5. The method according to claim 1, wherein the increase in the lipid content occurs at a constant growth rate of the organism.

6. The method according to claim 1, wherein the modulation of the expression and/or function of a lipase in the organism is achieved by inhibiting the expression and/or function of the lipase by the introduction and expression of an RNA interference construct in the organism, which is directed against the sequence of the lipase.

7. The method according to claim 6, wherein the RNA interference construct comprises a sequence that is at least 50% identical to one of SEQ ID NOs: 1 to 10.

8. A process for the production of an organism with an increased lipid content, which comprises carrying out the process according to claim 1.

9. An organism, produced by the method according to claim 8.

10. A method for improving the cultivation characteristics of an organism, comprising the modification of expression and/or function of a lipase in the organism, wherein the lipase is encoded by a nucleic acid sequence, comprising
iv. a sequence selected from SEQ ID NOs: 1 to 10, or comprising
v. a sequence of at least 50% sequence identity to one of SEQ ID NOs: 1 to 10, or comprising
vi. a sequence which hybridizes under standard conditions with a sequence of SEQ ID NOs: 1 to 10.

11. A nucleic acid comprising (i) a sequence selected from SEQ ID NOs: 1 to 10, or comprising (ii) a sequence having at least 50% sequence identity to one of SEQ ID NOs: 1 to 10, or comprising (iii) a sequence which hybridizes under standard conditions to a sequence of SEQ ID NOs: 1 to 10.

12. A vector comprising a nucleic acid according to claim 11.

13. A host cell containing a nucleic acid according to claim 11.

14. A lipase, having an amino acid sequence encoded by a nucleic acid according to claim 11.

15. A process for the extraction of lipids, comprising:
c. providing a culture of the organism according to claim 9,
d. expanding the culture until a desired culture density has been reached, and
e. extraction of the lipids from the culture.

16. A lipid mixture, obtained by the method according to claim 15.

17. The method, according to claim 2, wherein the organism is a Phaeodactylaceae.

18. The method, according to claim 17, wherein the organism is Phaeodactylum tricornutum.

19. The method, according to claim 14, wherein the lipid content is increased in the organism 150% to 200%.

20. The method, according to claim 7, wherein the RNA interference construct comprises SEQ ID NO: 10.
